(19) United States
(12) Patent Application Publication
(10) Pub. No.: US 2025/0127850 A1
(43) Pub. Date: Apr. 24, 2025

(54) METHODS AND COMPOSITIONS FOR INHIBITING FIBROSIS AND SCARRING

(71) Applicant: The University of North Carolina at Chapel Hill, Chapel Hill, NC (US)

(72) Inventors: Hua Mei, Cary, NC (US); Chi Hao Tsai, Chapel Hill, NC (US)

(21) Appl. No.: 18/688,256

(22) PCT Filed: Sep. 1, 2022
(86) PCT No.: PCT/US2022/075814

§ 371 Date: Feb. 29, 2024

Related U.S. Application Data
(60) Provisional application No. 63/239,678, filed on Sep. 1, 2021.

(51) Int. Cl. A61K 38/17; A61P 11/00; A61P 17/02

(52) U.S. Cl. CPC A61K 38/1709; A61P 11/00; A61P 17/02

(57) ABSTRACT

This invention relates to methods and compositions for inhibiting fibrosis and the formation of scars, e.g., as a result of corneal wounds, by administering NBL1 to a subject. The invention further relates to methods and compositions for inhibiting the growth of developing scars by administering NBL1 to the scar. The invention also relates to methods and compositions for facilitating wound re-epithelialization and partially restoring corneal stromal thickness by administering NBL1 to the wounded cornea.

### STATEMENT OF PRIORITY

This application claims the benefit of U.S. Provisional Application Ser. No. 63/239,678, filed Sep. 1, 2021, the entire contents of which are incorporated by reference herein.

### FIELD OF THE INVENTION

This invention relates to methods and compositions for inhibiting fibrosis and the formation of scars, e.g., as a result of corneal wounds, by administering NBL1 to a subject. The invention further relates to methods and compositions for inhibiting the growth of developing scars by administering NBL1 to the scar. The invention also relates to methods and compositions for facilitating wound re-epithelialization and partially restoring corneal stromal thickness by administering NBL1 to the wounded cornea.

### BACKGROUND OF THE INVENTION

Fibrosis affects nearly all tissues and organ systems. Disorders are associated with and caused by excessive scarring and/or adhesions resulting from surgery, chemotherapeutic drug-induced fibrosis, radiation-induced fibrosis, and injuries and burns. Fibrotic tissue remodeling can also influence cancer metastasis and accelerate chronic graft rejection in transplant recipients. Fibrosis is also a component of the foreign body reaction to implanted devices. Diseases in which fibrosis is a major cause of morbidity and mortality include interstitial lung diseases, liver cirrhosis, liver fibrosis resulting from chronic hepatitis B or C infection, kidney disease, heart disease, and systemic sclerosis. Fibroproliferative disorders also include systemic and local scleroderma, keloids and hypertrophic scars, atherosclerosis, restenosis, and eye diseases including corneal scarring, macular degeneration, and retinal and vitreal retinopathy.

Fibrosis is central to the pathogenesis of many chronic lung disorders, including asthma, pneumoconioses, and many infections. The quintessential fibrotic lung diseases, however, are the fibrotic interstitial lung diseases, usual interstitial pneumonia (UIP) and fibrotic variant of non-specific interstitial pneumonia (NSIP). These illnesses are of unknown cause and are characterized by progressive lung fibrosis, typically culminating in respiratory failure and premature death. No treatment has been clearly effective in altering the clinical course of these diseases.

Corneal scarring is one of the major causes of blindness and affects more than 4 million people worldwide (Nischal, Natl. Med. J. India 32(6): 383 (2019)). Common causes of corneal scars include infection, injury, and ocular surgery. Corneal wounds on the epithelial layer heal without a scar. When the wound reaches the stroma, wound repair causes scar formation following three overlapping phases: inflammation, proliferation/re-epithelialization, and remodeling (Darby et al., Cosmet. Investig. Dermatol. 7: 301 (2014); desJardins-Park et al., Front. Physiol. 2019. 10:322 (2019); Wilson et al., J. Funct. Biomater. 3(3):642 (2012)). There is no drug to treat or to prevent corneal scars. The current clinical approach to minimize corneal scar formation is to reduce inflammation by topical application of corticosteroids. However, the anti-scar effect of this therapy is controversial. Some clinical trials showed that topical application of corticosteroids had no beneficial or harmful effect on rescuing the best-corrected visual acuity (Palioura et al., Clin. Ophthalmol. 10:179 (2016); Srinivasan et al., Am. J. Ophthalmol. 157(2):327 (2014)). In nocardia-related ulcers, corticosteroid use may lead to larger scars, probably due to recurrent infections and delayed epithelial wound closure (Srinivasan et al., Am. J. Ophthalmol. 157(2):327 (2014); Kwok et al., Eye Contact Lens 45(6):347 (2019)). Additional potential future treatments to reduce corneal scars include limbus-derived stem cell therapy (under clinical trial) (Basu et al., Sci. Transl. Med. 6(266):266ra172 (2014)).

The present invention overcomes shortcomings in the art by providing effective treatments that reduce fibrosis, adhesion formation, and scar formation, e.g., corneal scar formation, facilitate wound re-epithelialization, and prevent the loss of fibroblast cells during fibrosis.

### SUMMARY OF THE INVENTION

The present invention is based, in part, on the development of compositions and methods using NBL1 to inhibit fibrosis. NBL1 facilitates re-epithelization and healing of corneal wounds and reduces the formation of myofibroblasts to minimize scar formation and has an anti-fibrotic effect on other primary fibroblasts isolated from different organs from healthy human donors. The methods are also useful for inhibiting growth of existing fibrosis, adhesions, and scars as well as diminishing existing fibrosis, adhesions, and scars. Importantly, the application of NBL1 to corneal wounds restores both mechanical strength and transparency to the cornea. This effect of NBL1 on corneal tissue is a previously undescribed biological function of the protein. NBL1 may be one of the first agents effective to reduce corneal scars and improve vision.

Thus, one aspect of the invention relates to a method for preventing or attenuating fibrosis and/or adhesion formation and/or scar formation in a subject in need thereof, the method comprising administering to the subject an effective amount of NBL1, thereby preventing or attenuating fibrosis and/or adhesion formation and/or scar formation.

Another aspect of the invention relates to a method of inhibiting growth of an existing fibrosis, adhesion, or scar in a subject, comprising contacting the fibrosis, adhesion, or scar with an effective amount of NBL1, thereby inhibiting growth of the fibrosis, adhesion, or scar.

A further aspect of the invention relates to a method of diminishing a fibrosis, adhesion, or scar in a subject, comprising contacting the fibrosis, adhesion, or scar with an effective amount of NBL1, thereby diminishing the fibrosis, adhesion, or scar.

An additional aspect of the invention relates to a method for treating, preventing, or inhibiting progression of a fibrotic disease in a subject in need thereof, the method comprising administering to the subject a composition comprising an effective amount of NBL1 to the site of fibrosis, thereby treating, preventing, or inhibiting progression of the fibrotic disease.

Another aspect of the invention relates to a method of treating a corneal wound in a subject to facilitate wound re-epithelization, comprising contacting the corneal wound with a therapeutically effective amount of NBL1, thereby facilitating wound re-epithelization.

A further aspect of the invention relates to a method of treating a corneal wound in a subject to minimize scarring, comprising contacting the corneal wound with a therapeutically effective amount of NBL1, thereby treating the corneal wound.

Another aspect of the invention relates to a method of inhibiting scar formation in a corneal wound in a subject, comprising contacting the corneal wound with a therapeutically effective amount of NBL1, thereby inhibiting scar formation in the corneal wound.

An additional aspect of the invention relates to a method of inhibiting growth of an existing scar in a cornea in a subject, comprising contacting the scar with a therapeutically effective amount of NBL1, thereby inhibiting growth of the scar.

A further aspect of the invention relates to a method of diminishing a corneal scar in a subject, comprising contacting the scar with a therapeutically effective amount of NBL1, thereby diminishing the scar.

The methods of the invention may be carried by contacting a corneal wound or a corneal scar with a NBL1 protein, a nucleic acid encoding NBL1, and/or a cell recombinantly altered to express NBL1.

Another aspect of the invention relates to a pharmaceutical composition comprising NBL1 protein and a pharmaceutically acceptable carrier, wherein the pharmaceutical composition is suitable for ophthalmic administration.

An additional aspect of the invention relates to a pharmaceutical composition comprising a nucleic acid encoding NBL1 protein and a pharmaceutically acceptable carrier, wherein the pharmaceutical composition is suitable for ophthalmic administration.

A further aspect of the invention relates to a pharmaceutical composition comprising a cell recombinantly altered to express NBL1 and a pharmaceutically acceptable carrier, wherein the pharmaceutical composition is suitable for ophthalmic administration.

These and other aspects of the invention are set forth in more detail in the description of the invention below.

### BRIEF DESCRIPTION OF THE DRAWINGS

FIGS. 1A-IC show corneal stromal cells were activated to secrete a significantly higher level of NBL1 during non-scarring wounding healing and scarring wound healing in mouse and human corneas. The inventors previously established a non-scarring corneal wound healing model in mice, in which the corneal epithelial wound healed without scar, evidenced by the minimal expression of Acta2 (α-SMA gene) at mRNA level (A) and at protein level (B) in wounded cornea compared to that in unwounded cornea. A list of genes was generated ranking from high to low on the differential expression in wounded corneas compared to unwounded corneas. NBL1 (DAN family member) was on top of the list showing one of the highest level of differential expression. The rest of the DAN family members showed minimal expression in unwounded and wounded corneas (A). In human corneas undergoing a scarring wound healing using an organ culture model, NBL1 was highly expressed in activated corneal stromal cells and in myofibroblasts compared to the low expression in unwounded human corneas (C). White arrowhead points to the wound cut by trephine. Scale bars in (B) and (C) represent 50 and 200 μm respectively. White circle indicates the activated stromal cells expressing a high level of NBL1. White dashed circle indicates the myofibroblasts expressing a high level of NBL1.

FIGS. 2A-2C show the purchased NBL1-Fc chimeric protein was biologically active as a BMP antagonist in mouse 3T3-J2 cells and in human HEK293 cells. Both cell lines were serum starved for 1 hour followed by treatment indicated in figure: control (vehicle only), BMP4 (50 ng/ml), BMP4+1×NBL1 (3 μg/ml), BMP+1×Fc (same molar concentration as 1×NBL1), BMP4+10×NBL1 (30 μg/ml), BMP+10×Fc (same molar concentration as 10×NBL1) for 4 h before examination by immunocytochemistry (ICC). (A) Representative pictures of the ICC data in 3T3-J2 cells and HEK293 cells. (B) Quantitation of the ICC data in 3T3-J2 cells. (C) Quantitation of the ICC data in HEK293 cells. Scale bars represent 100 μm. N=4. *: p<0.05.

FIGS. 3A-3J show NBL1 treatment facilitated corneal wound re-epithelization, partially restored corneal stromal thickness, and reduced corneal scar formation after mechanical wound on mouse corneas. (A) NBL1 treatment at both 1×(3 μg/ml) and 10×(30 μg/ml) showed a faster wound re-epithelization revealed by fluorescein staining. (B) Representative pictures showing that NBL1 reduced corneal hyper-reflectivity (indicative of scar) revealed by OCT. (C) Quantitative analysis of the OCT data showing that NBL1 reduced the hyper-reflective area indicating the size of the corneal scar. (D) Quantitative analysis of the OCT data showing that NBL1 partially restored corneal stromal thickness after a 2-week treatment. (E) Representative pictures of intact mouse eyes and dissected mouse corneas showing that NBL1 reduced corneal opacity after wounding. Red circle indicates the edge of dissected cornea. (F) Quantitative analysis of the corneal opacity based on the double-blind observation and grading from 3 independent researchers. Corneal opacity: 1/clear, 2/slightly cloudy, 3/very cloudy, and 4/opaque. (G) Representative pictures showing the expression of β-SMA (myofibroblast marker) is reduced in NBL1-treated corneas compared to the vehicle (Ctl) and the Fc-treated corneas using IHC. (H) Quantitative analysis of the IHC data on the percentage of the α-SMA⁺ area in the central corneal stroma. (I) Representative pictures showing the expression of fibronectin (scar deposition) is reduced in NBL1-treated corneas compared to the vehicle (Ctl) and the Fc-treated corneas using IHC. (J) Quantitative analysis of the IHC data on the percentage of the fibronectin⁺ area in the central corneal stroma. Scale bars represent 50 μm. N=6-8. *: p<0.05.

FIG. 4 shows representative pictures showing the wound re-epithelization in mouse corneas after mechanical wounding in different treatment groups.

FIG. 5 shows representative pictures showing the formation of corneal scars in mouse eyes after mechanical wounding in different treatment groups during the 2-week study.

FIGS. 6A-6B show NBL1 reduced tissue fibrosis in human corneas using an organ culture model. Pairs of human corneas were induced to form a scar in culture by mechanical wounding and incubation in 1 ng/ml TGF-β1 for 2 weeks. NBL1 treated human corneas showed a significantly reduced number of myofibroblasts (α-SMA) with representative pictures in (A) and quantitative analysis in (B). N=4 pairs of human corneas. White arrowhead points to the wound cut by trephine. Scale bars represent 10 μm. N=4. *: p<0.05.

FIGS. 7A-7B show NBL1 rescued cell loss of primary lung fibroblast cells caused by TGF-β1 in serum-free medium. (A) Representative pictures showing the number of primary lung fibroblast cells from healthy human donors after a 6-day culture. (B) Cell number collected per well. Scale bars represent 10 μm. N=6. *: p<0.05.

FIGS. 8A-8B show NBL1 showed an anti-fibrotic effect on primary lung fibroblasts from healthy human donors. (A) Representative pictures showing α-SMA (fibrotic marker) expression in different treatment groups after a 6-day culture. (B) Quantitative analysis on the α-SMA expression in different treatment groups. Scale bars represent 10 μm. N=6. *: p<0.05.

FIGS. 9A-9B show NBL1 showed an anti-fibrotic effect on primary dermal fibroblasts from healthy human donors. (A) Representative pictures showing α-SMA (fibrotic marker) expression in different treatment groups after 2-day culture. (B) Quantitative analysis on the α-SMA expression in different treatment groups. Scale bars represent 10 μm. N=5. p<0.05.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of rAAV constructs, modified capsid proteins, packaging vectors expressing the AAV rep and/or cap sequences, and transiently and stably transfected packaging cells. Such techniques are known to those skilled in the art. See, e.g., SAMBROOK et al., MOLECULAR CLONING: A LABORATORY MANUAL 4th Ed. (Cold Spring Harbor, NY, 2012); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the term “polypeptide” encompasses both peptides and proteins, unless indicated otherwise.

A “polynucleotide,” “nucleic acid,” or “nucleotide sequence” may be of RNA, DNA or DNA-RNA hybrid sequences (including both naturally occurring and non-naturally occurring nucleotides), but is preferably either a single or double stranded DNA sequence.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc. The region in a nucleic acid sequence or polynucleotide in which one or more regulatory elements are found may be referred to as a “regulatory region.”

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

The term “open reading frame (ORF),” as used herein, refers to the portion of a polynucleotide (e.g., a gene) that encodes a polypeptide, and is inclusive of the initiation start site (i.e., Kozak sequence) that initiates transcription of the polypeptide. The term “coding region” may be used interchangeably with open reading frame.

The term “codon-optimized,” as used herein, refers to a gene coding sequence that has been optimized to increase expression by substituting one or more codons normally present in a coding sequence (for example, in a wildtype sequence, including, e.g., a coding sequence for NBL1) with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry and/or initiation sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell. Codon-optimization also provides the ability to distinguish a codon-optimized gene and/or corresponding mRNA from an endogenous gene and/or corresponding mRNA in vitro or in vivo.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989). Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene/ORF. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

As used herein, the term “modified,” as applied to a polynucleotide or polypeptide sequence, refers to a sequence that differs from a wildtype sequence due to one or more deletions, additions, substitutions, or any combination thereof.

As used herein, by “isolate” (or grammatical equivalents) a virus vector, it is meant that the virus vector is at least partially separated from at least some of the other components in the starting material.

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

As used herein, the term “prevent,” “prevents,” or “prevention” (and grammatical equivalents thereof) means to delay or inhibit the onset of a disease. The terms are not meant to require complete abolition of disease, and encompass any type of prophylactic treatment to reduce the incidence of the condition or delays the onset of the condition.

A “treatment effective,” “therapeutically effective,” or “effective” amount as used herein is an amount that is sufficient to provide some improvement or benefit to the subject. Alternatively stated, a “treatment effective,” “therapeutically effective,” or “effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

By “substantially retain” a property, it is meant that at least about 75%, 85%, 90%, 95%, 97%, 98%, 99% or 100% of the property (e.g., activity or other measurable characteristic) is retained.

The terms “fibrotic” disease or a “fibroproliferative” disease refers to a disease characterized by scar formation and/or the over production of extracellular matrix by connective tissue. Fibrotic disease occurs as a result of tissue damage. It can occur in virtually every organ of the body. Examples of fibrotic or fibroproliferative diseases include, but are not limited to, pulmonary fibrosis, idiopathic pulmonary fibrosis, fibrotic interstitial lung disease, interstitial pneumonia, fibrotic variant of non-specific interstitial pneumonia, cystic fibrosis, lung fibrosis, silicosis, asbestosis, asthma, chronic obstructive pulmonary lung disease (COPD), pulmonary arterial hypertension, liver fibrosis, liver cirrhosis, renal fibrosis, glomerulosclerosis, diabetic nephropathy, heart disease, fibrotic valvular heart disease, systemic fibrosis, rheumatoid arthritis, excessive scarring resulting from surgery or other injury, adhesions, chemotherapeutic drug-induced fibrosis, radiation-induced fibrosis, macular degeneration, retinal and vitreal retinopathy, atherosclerosis, and restenosis. Fibrotic disease or disorder, fibroproliferative disease or disorder and fibrosis are used interchangeably herein.

The terms “patient,” “subject,” “individual,” and the like are used interchangeably herein, and refer to any animal, or cells thereof whether in vitro or in situ, amenable to the methods described herein. In one embodiment, the patient, subject, or individual is a mammal. In some embodiments, the mammal is a mouse, a rat, a guinea pig, a non-human primate, a dog, a cat, or a domesticated animal (e.g., horse, cow, pig, goat, sheep). In certain embodiments, the patient, subject or individual is a human.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible level or activity (at most, an insignificant amount, e.g., less than about 10% or even 5%).

The term “contact” or grammatical variations thereof as used with respect to a polypeptide and a receptor, refers to bringing the polypeptide and the receptor in sufficiently close proximity to each other for one to exert a biological effect on the other. In some embodiments, the term contact means binding of the polypeptide to the receptor.

The term “decreasing risk” refers to lowering the likelihood of establishing a disease, disorder, or condition and/or decreasing the severity or extent of a disease, disorder or condition if it is established.

The term “administering” or “administration” of an agent or drug to a subject includes any route of introducing or delivering to a subject a compound to perform its intended function. Administration can be carried out by any suitable route, including orally, intranasally, by inhalation, or parenterally (intravenously, intramuscularly, intraperitoneally, subdermally, or subcutaneously). Administration includes self-administration and the administration by another.

It is also to be appreciated that the various modes of treatment or prevention of medical diseases and conditions as described are intended to mean “substantial,” which includes total but also less than total treatment or prevention, and wherein some biologically or medically relevant result is achieved.

### Methods of Inhibiting Fibrosis, Adhesion Formation, and Scar Formation

One aspect of the invention relates to a method for preventing or attenuating fibrosis and/or adhesion formation and/or scar formation in a subject in need thereof, the method comprising administering to the subject an effective amount of neuroblastoma suppressor of tumorigenicity 1 (NBL1), thereby preventing or attenuating fibrosis and/or adhesion formation and/or scar formation.

Another aspect of the invention relates to a method of inhibiting growth of an existing fibrosis, adhesion, or scar in a subject, comprising contacting the fibrosis, adhesion, or scar with an effective amount of NBL1, thereby inhibiting growth of the fibrosis, adhesion, or scar.

A further aspect of the invention relates to a method of diminishing a fibrosis, adhesion, or scar in a subject, comprising contacting the fibrosis, adhesion, or scar with an effective amount of NBL1, thereby diminishing the fibrosis, adhesion, or scar.

In some embodiments, the subject has a wound, e.g., a cut, scratch, scrape, incision, burn, etc. In some embodiments, the NBL1 is administered during or immediately after surgery, e.g., intra-abdominal surgery or intra-thoracic surgery, e.g., to prevent or minimize fibrosis and adhesions. In some embodiments, the NBL1 is administered to the subject to prevent or minimize formation of adhesions at a site of implantation, e.g., of a device. In some embodiments, the implanted device is coated or sprayed with the NBL1 prior to, during, or after implantation. In some embodiments, the NBL1 is administered directly to the subject's tissue(s) (e.g., by spray, topical administration (e.g., direct application to internal tissues, including the peritoneal cavity), injection, and the like). In some embodiments, the NBL1 is administered at the time of implantation or surgery. In some embodiments, the NBL1 is administered at the time of or soon after injury.

The NBL1 protein may be a human NBL1 protein or a functional fragment thereof. The amino acid sequence and nucleic acid sequence of human NBL1 is well-known in the art and can be found, e.g., in GenBank Accession No. AY049783.1, incorporated by reference herein in its entirety. In some embodiments, the NBL1 protein has an amino acid sequence at least 80% identical to the sequence disclosed in GenBank Accession No. AY049783.1, e.g., at least 80%, 85%, 90%, 95%, 96%, 97%, 98%, or 99% identical.

In some embodiments, the NBL1 protein may be modified or derivatized. As used herein, the term “derivative” is used to refer to a polypeptide which differs from a naturally occurring NBL1 by minor modifications to the naturally occurring polypeptide, but which significantly retains a biological activity of NBL1. Minor modifications include, without limitation, changes in one or a few amino acid side chains, changes to one or a few amino acids (including deletions, insertions, and/or substitutions), changes in stereochemistry of one or a few atoms (e.g., D-amino acids), and minor derivatizations, including, without limitation, methylation, glycosylation, phosphorylation, acetylation, myristoylation, prenylation, palmitation, amidation, and addition of glycosylphosphatidyl inositol. The term “substantially retains,” as used herein, refers to a fragment, derivative, or other variant of a polypeptide that retains at least about 20% of the activity of the naturally occurring polypeptide (e.g., inhibiting fibrosis), e.g., about 30%, 40%, 50%, 60%, 70%, 80% or more. In some embodiments, the derivative of NBL1 contains mutations (deletions, insertions, and/or substitutions in any combination) of 10 or fewer amino acid residues, e.g., 10, 9, 8, 7, 6, 5, 4, 3, or 2 or fewer mutations. In some embodiments, the NBL1 derivative comprises an amino acid sequence that is at least about 80%, 85%, 90%, 91%, 92%, 93%, 94%, 95%, 96%, 97%, 98%, or 99% identical to the amino acid sequence of GenBank Accession No. AY049783.1.

In some embodiments, the NBL1 can be modified for in vivo use by the addition, at the amino- and/or carboxyl-terminal ends, of a blocking agent to facilitate survival of the relevant polypeptide in vivo. This can be useful in those situations in which the peptide termini tend to be degraded by proteases. Such blocking agents can include, without limitation, additional related or unrelated peptide sequences that can be attached to the amino and/or carboxyl terminal residues of the protein to be administered. This can be done either chemically during the synthesis of the protein or by recombinant DNA technology by methods familiar to artisans of average skill. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the proteins can be covalently or noncovalently coupled to pharmaceutically acceptable “carrier” proteins prior to administration, e.g., as a fusion protein. In some embodiments, the carrier protein is the Fc region of an antibody, e.g., a human IgG Fc region, e.g., a fusion protein comprising NBL1 and Fc.

The NBL1 can be produced by any suitable method. In some embodiments, the NBL1 is produced recombinantly using methods well known in the art and as described herein.

In some embodiments, a nucleic acid encoding NBL1 protein may be delivered to the subject, e.g., to a wound, e.g., by directly contacting the wound with a composition comprising the nucleic acid. The nucleic acid may be part of a vector, e.g., a non-viral (e.g., plasmid) or viral (e.g., adeno-associated virus, retrovirus, lentivirus, poxvirus, alphavirus, baculovirus, vaccinia virus, herpes virus, Epstein-Barr virus, or adenovirus) vector. In some embodiments, the nucleic acid encoding NBL1 is operably linked to a promoter, e.g., a constitutive or regulatable promoter. In some embodiments, the vector may be one that expresses NBL1 for a limited time, e.g., 1, 2, 3, or 4 weeks, or 1, 2, 3, 4, 5, or 6 months. In some embodiments, the promoter is a regulatable promoter and expression of NBL1 protein is regulated to occur for a specific amount of time, e.g., 1, 2, 3, or 4 weeks, or 1, 2, 3, 4, 5, or 6 months.

In some embodiments, a cell recombinantly altered to express NBL1 protein may be delivered to the subject, e.g., to the wound, e.g., by directly contacting the wound with a composition comprising the cell. The cell may be any suitable cell type, e.g., an epithelial cell, a stromal cell, a progenitor cell, or a stem cell. The cell may be autologous. The cells may be recombinantly altered to incorporate a nucleic acid expressing NBL1, e.g., in the genome or episomally. In some embodiments, the cells are designed to express NBL1 for a limited time, e.g., 1, 2, 3, or 4 weeks, or 1, 2, 3, 4, 5, or 6 months. In other embodiments, the cells are designed to survive for a limited time, e.g., 1, 2, 3, or 4 weeks, or 1, 2, 3, 4, 5, or 6 months.

In certain embodiments, the NBL1 (whether in the form of a protein, nucleic acid, or cell) is delivered to the subject as soon as possible after wound formation. In some embodiments, the wound is contacted with NBL1 less than 48 hours after wound formation, e.g., less than 24 hours or less than 12, 11, 10, 9, 8, 7, 6, 5, 4, 3, or 2 hours.

The NBL1 may be administered in any amount and schedule shown to be effective to facilitate re-epithelization and minimize adhesion and/or scar formation. In some embodiments, the wound is contacted with NBL1 once every 7 days, e.g., once every 6, 5, 4, 3, 2, or 1 day. In some embodiments, the administration is continued until for as long as necessary to minimize adhesion and/or scar formation, e.g., until continued growth of the adhesion and/or scar stops. In certain embodiments, administration is continued for 1, 2, 3, or 4 weeks or 2, 3, 4, 5, or 6 months.

In some embodiments with existing fibrosis or an existing adhesion or scar, the fibrosis, adhesion, or scar is contacted with NBL1 less than 1 month after fibrosis, adhesion, or scar formation, e.g., less than 3, 2, or 1 week. In some embodiments, the fibrosis, adhesion, or scar is contacted with NBL1 once every 7 days, e.g., once every 6, 5, 4, 3, 2, or 1 day.

A further aspect of the invention relates to a method for treating, preventing, or inhibiting progression of a fibrotic disease in a subject in need thereof, the method comprising administering to the subject a composition comprising an effective amount of NBL1 to the site of fibrosis, thereby treating, preventing, or inhibiting progression of the fibrotic disease.

In one aspect, this invention relates to a method for treating a fibrotic lung disease in a subject in need thereof, the method comprising administering to the patient an effective amount (e.g., a therapeutically or prophylactically effective amount) of NBL1.

In some embodiments, the fibrotic lung disease may be selected from pulmonary fibrosis, fibrotic interstitial lung disease, interstitial pneumonia, fibrotic variant of non-specific interstitial pneumonia, cystic fibrosis, lung fibrosis, chronic obstructive pulmonary lung disease (COPD), or pulmonary arterial hypertension.

A specific set of diseases that are known to involve unchecked fibrotic response are idiopathic interstitial pneumonias, a diverse group of chronic pulmonary diseases characterized by varying levels of pulmonary fibrosis. The major factors driving the dominant pulmonary fibrotic response associated with various histologically distinct forms of idiopathic interstitial pneumonia (IIP) remains poorly defined, thereby contributing to the lack of effective clinical treatments for these diseases (Green, Overview of pulmonary fibrosis. Chest 2002; 122 (suppl. 6):334S-9S). Although many of these diseases exhibit a fibroproliferative response in the alveolar microenvironment leading to respiratory impairment, the degree of fibrotic change varies considerably among them (Nicholason, Am. J. Resp. Crit. Car Med/2000:162:2213-7; Chapman, J. Clin., Invest., 2004; 113:148-57). Equally perplexing is the clear demonstration that anti-inflammatory agents provide therapeutic benefit in less severe forms of IIP, such as non-specific interstitial pneumonia (NSIP) and respiratory bronchiolitis/interstitial lung disease (RBILD), but they often fail to prevent respiratory failure in patients with the most severe and deadly form of IIP—usual interstitial pneumonia (UIP) (Flaherty et al., Am. J. Med., 110:278-282 (2001); Lynch et al., Curr. Opin. Pulm. Med., 7:298-308 (2001); Flaherty et al., Thorax, 58:143-148 (2003)).

The fibrosis diseases treatable by NBL1 may be any fibrosing disorder, including, but not limited to one that is selected from the group consisting of pulmonary fibrosis, chronic obstructive pulmonary disease, hepatic fibrosis, rheumatoid arthritis, chronic renal disease, hypersensitivity pneumonitis, respiratory bronchiolitis/interstitial lung disease, Schistosoma mansoni infection, primary pulmonary hypertension (prevention of the formation of the plexiform lesion) herpes virus associated-diseases, which include lung and dermatological manifestations, keloid scarring, lupus, nephrogenic fibrosing dermopathy, fibrosing lesions associated with Schistosoma japonicum infection, autoimmune diseases, pathogenic fibrosis, Lyme disease, stromal remodeling in pancreatitis and stromal fibrosis, uterine fibroids, ovarian fibrosis, corneal fibrosis, congestive heart failure and other post-ischemic conditions, post-surgical scarring including abdominal adhesions, wide angle glaucoma trabeculotomy, or any combination thereof.

Pulmonary fibrosis is a common consequence and often a central feature of many lung diseases. In some disorders, fibrosis develops focally and to a limited degree. For example, in asthma and chronic obstructive pulmonary disease fibrotic changes occur around conducting airways where scarring may be important to the pathophysiology.

The diagnosis of these conditions can usually be made by careful history, physical examination, chest radiography, including a high resolution computer tomographic scan (HRCT), and open lung or transbronchial biopsies. However, in a significant number of patients, no underlying cause for the pulmonary fibrosis can be found. These conditions of unknown etiology have been termed idiopathic interstitial pneumonias. Histologic examination of tissue obtained at open lung biopsy allows classification of these patients into several categories, including Usual Interstitial Pneumonia (UIP), Desquamative Interstitial Pneumonia (DIP), and Non-Specific Interstitial Pneumonia (NSIP).

Idiopathic pulmonary fibrosis (IPF) is clinically a restrictive lung disease that characteristically progresses relentlessly to death from respiratory failure. Median survival of newly diagnosed patients with IPF is about 3 years. The quality of life for IPF patients is also poor. Despite this, there has been remarkably little progress in development and/or assessment of therapeutic strategies for IPF.

Pulmonary function tests may be employed to detect physiological changes associated with the presence of pulmonary disease. Pulmonary function tests performed in a clinical setting may be used to evaluate lung mechanics, gas exchange, pulmonary blood flow, and blood gases and pH. They are used to evaluate patients in the diagnosis of pulmonary disease, assessment of disease development, or evaluation of the risk of pulmonary complications from surgery.

Pulmonary function tests are used to indicate a battery of studies or maneuvers that may be performed using standardized equipment to measure lung function. Pulmonary function tests include simple screening spirometry, formal lung volume measurement, diffusing capacity for carbon monoxide, and arterial blood gases.

The pulmonary function tests may obtain such values as FEV (forced expiratory volume), FVC (forced vital capacity), FEF_{25%-75%} (forced expiratory flow rate), PEFR (peak expiratory flow rate), FRC (functional residual capacity), RV (residual volume), TLC (total lung capacity), and/or flow/volume loops. FEV measures the volume of air exhaled over a predetermined period of time by a forced expiration immediately after a full inspiration. FVC measures the total volume of air exhaled immediately after a full inspiration. FEF_{25%-75%} measures the rate of air flow during a forced expiration divided by the time in seconds for the middle half of expired volume. PEFR measures the maximum flow rate during a forced exhale starting from full inspiration. FRC is the volume of air remaining in the lungs after a full expiration. RV is the FRC minus the expiratory reserve volume. TLC is the total volume in the lungs at the end of a full inspiration. Flow/volume loops are graphical presentations of the percent of total volume expired (on the independent axis) versus the flow rate during a forced expiratory maneuver. Normal values and lower limits of normal can be determined as defined by Hankinson et al. (the National Health and Nutrition Examination Survey [NHANES]III predicted set).

The NBL1 may be NBL1 protein, a nucleic acid encoding NBL1, or a cell recombinantly altered to express NBL1 as described above.

In some embodiments, the NBL1 is administered systemically. In other embodiments, the NBL1 is administered locally, e.g., to a wound or a site of existing fibrosis or a site where fibrosis is likely to occur.

The NBL1 may be administered in a composition, e.g., a pharmaceutical composition. The composition may be, e.g., an injectable composition, a topical composition, or a sprayable composition.

### Methods of Inhibiting Corneal Scar Formation

The ability of NBL1 to facilitate re-epithelization of corneal wounds, promote healing, and inhibit fibrosis can be used advantageously to inhibit corneal scar formation. NBL1 speeds up the closure of the wound, produces smoother stroma borders, and increases stroma thickness. The application of NBL1 to corneal wounds restores mechanical strength and transparency to the injured cornea, thereby improving vision.

Thus, one aspect of the invention relates to a method of treating a corneal wound in a subject to facilitate wound re-epithelization, comprising contacting the corneal wound with a therapeutically effective amount of NBL1, thereby facilitating wound re-epithelization.

A further aspect of the invention relates to a method of treating a corneal wound in a subject to minimize scarring, comprising contacting the corneal wound with a therapeutically effective amount of NBL1, thereby treating the corneal wound.

Another aspect of the invention relates to a method of inhibiting scar formation in a corneal wound in a subject, comprising contacting the corneal wound with a therapeutically effective amount of NBL1, thereby inhibiting scar formation in the corneal wound.

As used herein, the term “corneal wound” refers to disruption of the epithelial layer and at least part of the stromal layer of the cornea. The corneal wound may be due to injury, infection, surgery, or any other type of trauma.

Corneal scar formation can be quantitated by methods known in the art and as described herein, and can include optical coherence tomography (OCT), direct measurement of cornea transparency, and measurement of scar tissue (e.g., the presence of abnormal myofibroblasts, fibers, and extracellular matrix (ECM), e.g., using immunohistochemistry).

The corneal wound may be contacted by NBL1 by any means known in the art. In some embodiments, NBL1 protein may be delivered to the wound, e.g., by directly contacting the wound with a composition comprising NBL1 protein. In some embodiments, the NBL1 protein is delivered by intrastromal injection, subconjunctival injection, intracameral injection, intravitreal injection, and/or topical administration. The topical administration may involve the use of a suitable composition such as eye drops or eye gel.

The NBL1 protein may be a human NBL1 protein or a functional fragment thereof. The amino acid sequence and nucleic acid sequence of human NBL1 is well-known in the art and can be found, e.g., in GenBank Accession No. AY049783.1, incorporated by reference herein in its entirety. In some embodiments, the NBL1 protein has an amino acid sequence at least 80% identical to the sequence disclosed in GenBank Accession No. AY049783.1, e.g., at least 80%, 85%, 90%, 95%, 96%, 97%, 98%, or 99% identical.

In some embodiments, the NBL1 protein may be modified or derivatized. As used herein, the term “derivative” is used to refer to a polypeptide which differs from a naturally occurring NBL1 by minor modifications to the naturally occurring polypeptide, but which significantly retains a biological activity of NBL1. Minor modifications include, without limitation, changes in one or a few amino acid side chains, changes to one or a few amino acids (including deletions, insertions, and/or substitutions), changes in stereochemistry of one or a few atoms (e.g., D-amino acids), and minor derivatizations, including, without limitation, methylation, glycosylation, phosphorylation, acetylation, myristoylation, prenylation, palmitation, amidation, and addition of glycosylphosphatidyl inositol. The term “substantially retains,” as used herein, refers to a fragment, derivative, or other variant of a polypeptide that retains at least about 20% of the activity of the naturally occurring polypeptide (e.g., inhibiting fibrosis), e.g., about 30%, 40%, 50%, 60%, 70%, 80% or more. In some embodiments, the derivative of NBL1 contains mutations (deletions, insertions, and/or substitutions in any combination) of 10 or fewer amino acid residues, e.g., 10, 9, 8, 7, 6, 5, 4, 3, or 2 or fewer mutations. In some embodiments, the NBL1 derivative comprises an amino acid sequence that is at least about 80%, 85%, 90%, 91%, 92%, 93%, 94%, 95%, 96%, 97%, 98%, or 99% identical to the amino acid sequence of GenBank Accession No. AY049783.1.

In some embodiments, the NBL1 can be modified for in vivo use by the addition, at the amino- and/or carboxyl-terminal ends, of a blocking agent to facilitate survival of the relevant polypeptide in vivo. This can be useful in those situations in which the peptide termini tend to be degraded by proteases. Such blocking agents can include, without limitation, additional related or unrelated peptide sequences that can be attached to the amino and/or carboxyl terminal residues of the protein to be administered. This can be done either chemically during the synthesis of the protein or by recombinant DNA technology by methods familiar to artisans of average skill. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the proteins can be covalently or noncovalently coupled to pharmaceutically acceptable “carrier” proteins prior to administration, e.g., as a fusion protein. In some embodiments, the carrier protein is the Fc region of an antibody, e.g., a human IgG Fc region, e.g., a fusion protein comprising NBL1 and Fc.

The NBL1 can be produced by any suitable method. In some embodiments, the NBL1 is produced recombinantly using methods well known in the art and as described herein.

In some embodiments, a nucleic acid encoding NBL1 protein may be delivered to the wound, e.g., by directly contacting the wound with a composition comprising the nucleic acid. The nucleic acid may be part of a vector, e.g., a non-viral (e.g., plasmid) or viral (e.g., adeno-associated virus, retrovirus, lentivirus, poxvirus, alphavirus, baculovirus, vaccinia virus, herpes virus, Epstein-Barr virus, or adenovirus) vector. In some embodiments, the nucleic acid encoding NBL1 is operably linked to a promoter, e.g., a constitutive or regulatable promoter. In some embodiments, the vector may be one that expresses NBL1 for a limited time, e.g., 1, 2, 3, or 4 weeks, or 1, 2, 3, 4, 5, or 6 months. In some embodiments, the promoter is a regulatable promoter and expression of NBL1 protein is regulated to occur for a specific amount of time, e.g., 1, 2, 3, or 4 weeks, or 1, 2, 3, 4, 5, or 6 months.

In some embodiments, a cell recombinantly altered to express NBL1 protein may be delivered to the wound, e.g., by directly contacting the wound with a composition comprising the cell. The cell may be any suitable cell type, e.g., an epithelial cell, a stromal cell, a progenitor cell, or a stem cell. The cell may be autologous. The cells may be recombinantly altered to incorporate a nucleic acid expressing NBL1, e.g., in the genome or episomally. In some embodiments, the cells are designed to express NBL1 for a limited time, e.g., 1, 2, 3, or 4 weeks, or 1, 2, 3, 4, 5, or 6 months. In other embodiments, the cells are designed to survive for a limited time, e.g., 1, 2, 3, or 4 weeks, or 1, 2, 3, 4, 5, or 6 months.

In certain embodiments, the NBL1 (whether in the form of a protein, nucleic acid, or cell) is delivered to the corneal wound as soon as possible after wound formation. In some embodiments, the corneal wound is contacted with NBL1 less than 48 hours after wound formation, e.g., less than 24 hours or less than 12, 11, 10, 9, 8, 7, 6, 5, 4, 3, or 2 hours.

The NBL1 may be administered in any amount and schedule shown to be effective to facilitate re-epithelization and minimize scar formation. In some embodiments, the corneal wound is contacted with NBL1 once every 7 days, e.g., once every 6, 5, 4, 3, 2, or 1 day. In some embodiments, the administration is continued until for as long as necessary to minimize scar formation, e.g., until continued growth of the scar stops. In certain embodiments, administration is continued for 1, 2, 3, or 4 weeks or 2, 3, 4, 5, or 6 months.

### Methods of Inhibiting Growth of Existing Corneal Scars and Diminishing Existing Corneal Scars

The inventors have surprisingly shown that contacting existing corneal scars with NBL1 can inhibit the growth of scars and even diminish existing scars. The use of NBL1 may increase the transparency of scarred corneas and improve vision.

Thus, an additional aspect of the invention relates to a method of inhibiting growth of an existing scar in a cornea in a subject, comprising contacting the scar with a therapeutically effective amount of NBL1, thereby inhibiting growth of the scar.

A further aspect of the invention relates to a method of diminishing a corneal scar in a subject, comprising contacting the scar with a therapeutically effective amount of NBL1, thereby diminishing the scar.

As used herein, the term “corneal scar” refers to fibrotic tissue that forms on a corneal wound. The scar may distort or block vision. Diminishing a scar refers to diminishing the area, thickness, and/or opacity of the scar. Diminishing a scar can be determined by the methods for quantitating scars described above.

The corneal scar may be contacted by NBL1 by any means known in the art. In some embodiments, NBL1 protein may be delivered to the scar, e.g., by directly contacting the scar with a composition comprising NBL1 protein. In some embodiments, the NBL1 protein is delivered by intrastromal injection, subconjunctival injection, intracameral injection, intravitreal injection, and/or topical administration. The topical administration may involve the use of a suitable composition such as eye drops or eye gel.

The NBL1 protein may be a human NBL1 protein or a functional fragment, modification, or derivative thereof.

In some embodiments, a nucleic acid encoding NBL1 protein may be delivered to the scar, e.g., by directly contacting the scar with a composition comprising the nucleic acid. The nucleic acid may be part of a vector, e.g., a non-viral (e.g., plasmid) or viral (e.g., adeno-associated virus, retrovirus, lentivirus, poxvirus, alphavirus, baculovirus, vaccinia virus, herpes virus, Epstein-Barr virus, or adenovirus) vector. In some embodiments, the nucleic acid encoding NBL1 is operably linked to a promoter, e.g., a constitute or regulatable promoter. In some embodiments, the vector may be one that expresses NBL1 for a limited time, e.g., 1, 2, 3, or 4 weeks, or 1, 2, 3, 4, 5, or 6 months. Om some embodiments, the promoter is a regulatable promoter and expression of NBL1 protein is regulated to occur for a specific amount of time, e.g., 1, 2, 3, or 4 weeks, or 1, 2, 3, 4, 5, or 6 months.

In some embodiments, a cell recombinantly altered to express NBL1 protein may be delivered to the scar, e.g., by directly contacting the scar with a composition comprising the cell. The cell may be any suitable cell type, e.g., an epithelial cell, a stromal cell or a stem cell. The cell may be autologous. The cells may be recombinantly altered to incorporate a nucleic acid expressing NBL1, e.g., in the genome or episomally. In some embodiments, the cells are designed to express NBL1 for a limited time, e.g., 1, 2, 3, or 4 weeks, or 1, 2, 3, 4, 5, or 6 months. In other embodiments, the cells are designed to survive for a limited time, e.g., 1, 2, 3, or 4 weeks, or 1, 2, 3, 4, 5, or 6 months.

In certain embodiments, the NBL1 (whether in the form of a protein, nucleic acid, or cell) is delivered to the corneal scar as soon as possible after scar formation. In some embodiments, the corneal scar is contacted with NBL1 less than 1 month after scar formation, e.g., less than 3, 2, or 1 week.

The NBL1 may be administered in any amount and schedule shown to be effective to inhibit continued scar growth or diminish the scar. In some embodiments, the corneal scar is contacted with NBL1 once every 7 days, e.g., once every 6, 5, 4, 3, 2, or 1 day. In some embodiments, the administration is continued until for as long as necessary to m inhibit continued scar growth or diminish the scar, e.g., until continued improvement is no longer seen. In certain embodiments, administration is continued for 1, 2, 3, or 4 weeks or 2, 3, 4, 5, or 6 months.

### Subjects, Pharmaceutical Formulations, and Modes of Administration

The methods of the present invention find use in both veterinary and medical applications. Suitable subjects include avians, reptiles, amphibians, fish, and mammals. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for corneal wounds or scarring. As a further option, the subject can be a laboratory animal and/or an animal model of disease. Preferably, the subject is a human.

In particular embodiments, the present invention provides one or more pharmaceutical compositions comprising NB1 protein, a nucleic acid encoding NBL1, or a cell recombinantly altered to express NBL1 as described herein in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid.

By “pharmaceutically acceptable” it is meant a material that is not toxic or otherwise undesirable, i.e., the material may be administered to a subject without causing any undesirable biological effects.

By “suitable for ophthalmic administration” it is meant that the pharmaceutical composition can be safely delivered to the eye and the active agent can reach the desired location within the eye.

The amount of the disclosed compositions administered to a subject will vary from subject to subject, depending on the nature of the disclosed compositions and/or formulations, the species, gender, age, weight and general condition of the subject, the mode of administration, and the like. Effective dosages and schedules for administering the compositions may be determined empirically, and making such determinations is within the skill in the art. The dosage ranges for the administration of the disclosed compositions are those large enough to produce the desired effect (e.g., to inhibit scar formation or dimmish existing scars). The dosage should not be so large as to outweigh benefits by causing extensive or severe adverse side effects, such as unwanted cross-reactions, anaphylactic reactions, and the like, although some adverse side effects may be expected. The dosage can be adjusted by the individual clinician in the event of any counterindications. Generally, the disclosed compositions and/or formulations are administered to the subject at a dosage of NBL1 protein ranging from 0.1 ng to 1000 ng or an amount of nucleic acids or cells to produce that amount of NBL1 protein. Dosages above or below the range cited above may be administered to the individual subject if desired. The compositions can be administered in any herein disclosed pharmaceutical composition comprising a pharmaceutically acceptable carrier.

Having described the present invention, the same will be explained in greater detail in the following examples, which are included herein for illustration purposes only, and which are not intended to be limiting to the invention.

### EXAMPLES

### Example 1: Inhibition of Corneal Scar Formation

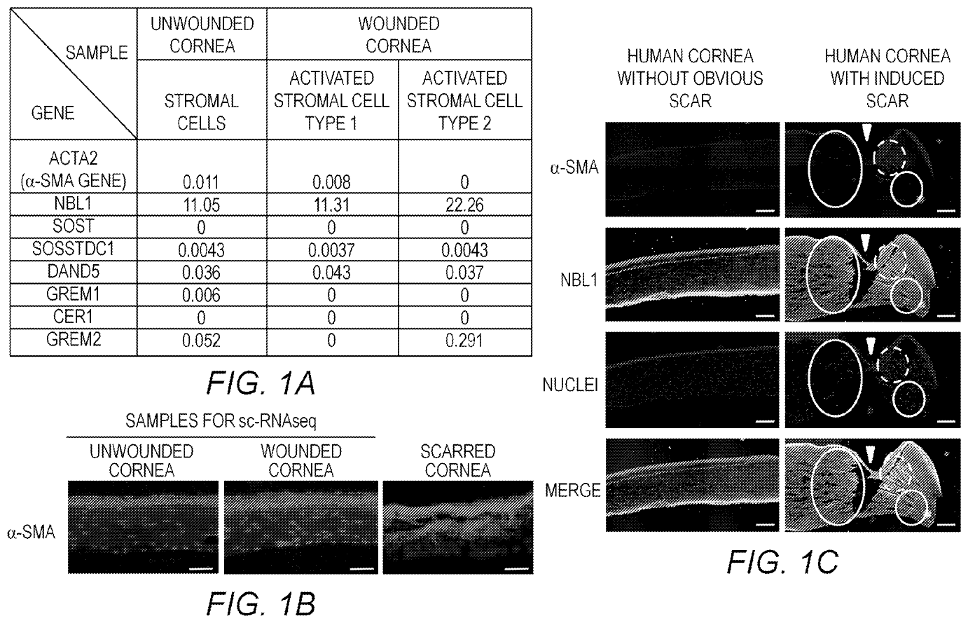

NBL1 was discovered from single-cell RNA sequencing (sc-RNAseq) data which were published previously (Song et al., Cells 11(13):1983 (2022)). A scarless corneal wound healing mouse model was established in which only corneal epithelium was removed without damaging the stroma using an ophthalmic tool called Algerbrush. Wounds restricted to the epithelial layer led to a scarless wound healing, which was confirmed by a low mRNA level (FIG. 1A) and protein level (FIG. 1B) of u-smooth muscle actin (α-SMA, myofibroblast and fibrosis marker) in wounded corneas. Unwounded corneas and scarred corneas caused by alkaline burn served as the negative and positive control respectively for the immunohistochemistry (IHC) (FIG. 1B). To explore how stromal cells were activated during the scarless wound healing, the genes that showed higher expression in the two types (approximately equal amount in cell numbers) of activated stromal cells in wounded corneas than the stromal cells in unwounded corneas were screened out. The activated genes during the scarless wound healing were ranked in a list from high to low in fold changes and NBL1 was one of the highest differentially expressed genes by activated corneal stromal cells during the scarless wound healing. As shown in FIG. 1A, NBL1 increased slightly in activated stromal cell type 1 (from 11.05 to 11.31 at logₑ scale) and increased significantly in activated stromal cell type 2 (from 11.05 to 22.26 at logₑ scale, which was >6000-fold change) compared to that in quiescent stromal cells in unwounded corneas. As NBL1 is a DAN family member, the other members in the DAN family were examined in the sc-RNAseq data and showed minimal expressions in unwounded and wounded corneas in mice (FIG. 1A), suggesting that NBL1 is the major BMP antagonist within the DAN family in cornea. To further explore whether NBL1 was highly secreted upon wounding in human corneas, a human organ culture model was employed in which corneas were wounded with a 4-mm trephine and incubated in medium containing 1 ng/ml TGF-β1 to induce a scarring wound healing. It was observed that wound-activated human corneas expressed a significantly higher level of NBL1 compared to the unwounded human corneas, especially around the wound area (FIG. 1C). Both activated stromal cells (white circle in FIG. 1C) and myofibroblasts (white dashed circle in FIG. 1C) expressed a high level of NBL1, suggesting an important role of NBL1 in corneal wound healing and scar formation in human.

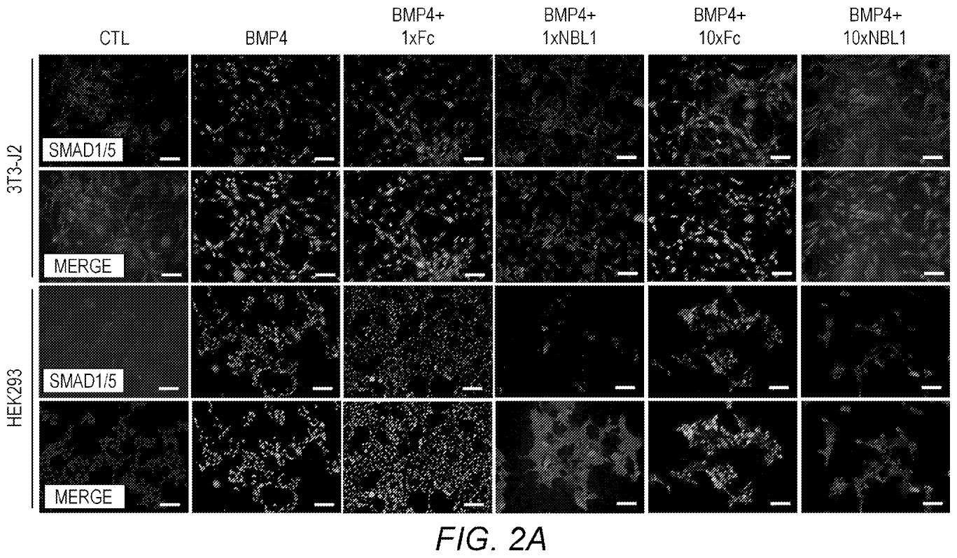

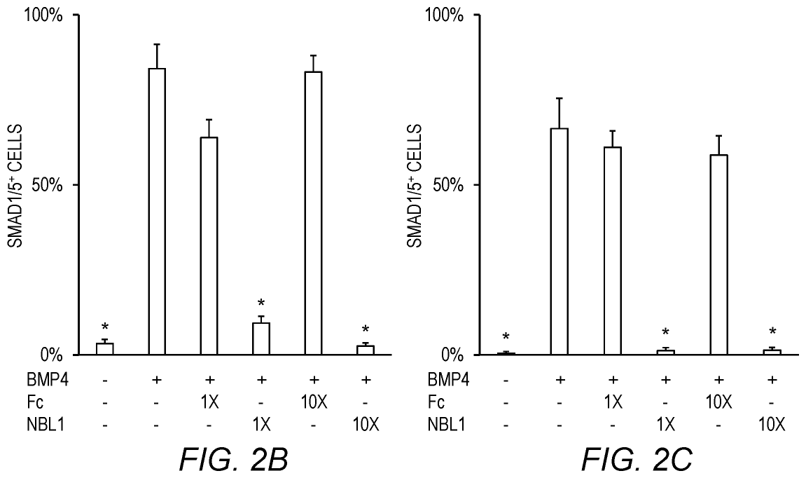

To examine the function of NBL1 in corneal wound healing, wounded corneas were treated with NBL1 using a mouse model and a human organ culture model. NBL1 was purchased from R&D Systems (955-DA), which is human NBL1 fused with a human IgG1 (Fc) tag. The Fc-fusion proteins are a widely used strategy of therapeutic protein design already approved by the FDA. Fc-fusion proteins have the advantage of protein dimerization and enhanced stability in vivo (Yang et al., Front. Immunol. 8:1860 (2017)); both protein dimerization (Gipson et al., Biochem. J. 477(17):3167 (2020)) and enhanced stability are crucial for proper NBL1 function in vivo, thus selected for this study. Human IgG1 Fc protein (R&D Systems, 110-HG) was used as a negative control (“Fc” in figures). The vehicle served as an additional control throughout the study (“Ctl” in figures). Before the mouse and human organ culture experiments, the purchased NBL1 fusion protein was confirmed to be biologically active in mouse and human cells even though its biological activity has been tested by the manufacturer. Both mouse (3T3-J2) and human (HEK293) cell lines were seeded at 3×10⁴ cells/well in a 4-well chamber slide overnight, followed by serum starvation for 1 hour. Then the cells were treated with control (“Ctl”, vehicle only), BMP4 (50 ng/ml, R&D Systems, 314-BPE), BMP4+1×NBL1(3 μg/ml), BMP+1×Fc (same molar concentration as 1×NBL1), BMP4+10×NBL1(30 μg/ml), and BMP+10×Fc (same molar concentration as 10×NBL1) for 4 h before examination by immunocytochemistry (ICC) (FIGS. 2A-2C). Data showed that BMP4 activated BMP signaling and induced phospho-Smad1/5 (Cell Signaling Technology, 9516T) expression and nuclear translocation, which was suppressed by NBL1, indicating that the purchased NBL1 protein was biologically active as a BMP antagonist in both mouse and human cells. Fc showed no effect on BMP4-induced phospho-Smad1/5 expression and nuclear translocation (FIGS. 2A-2C). After validating the bioactivity of the purchased protein NBL1, its function was examined on corneal wound healing and scar formation using an in vivo mouse model and in vitro human organ culture model.

Function of NBL1 on wounded mouse corneas (mouse model). C57BL/6J mice (the Jackson Laboratory) at around 2 months old with approximately equal number of male and female mice were used in this study. Mouse corneas were wounded by removing corneal epithelium and anterior stroma (around 50% of the total stromal thickness) mechanically using Algerbrush, followed by NBL1 treatment using intrastromal (first dose, injected immediately after wounding) and subconjunctival injections (following doses, injected every 3 days) for 2 weeks. Two doses of NBL1 were tested: (1) NBL1 at 3 ng/cornea/injection (NBL1 solution at 3 μg/ml and 1 μl was injected per time, named “1×NBL1”), and (2) NBL1 at 30 ng/cornea/injection (named “10×NBL1”). Treatment with Fc protein at corresponding molar concentrations (named “1×Fc” and “10×Fc”) served as control groups. Treatment with vehicle served as an additional control (named “Ctl”). The severity of corneal scar depends on the size and depth of the wound: a bigger and deeper wound causes more scarring (Wilson et al., J. Funct. Biomater. 3(3):642 (2012); Tuft et al., Br. J. Ophthalmol. 77(4):243 (1993)). Therefore, it is crucial to keep a consistent wounding among different groups. After practice, the inventors managed to remove consistently around 25-35% of corneal surface (examined by fluorescein staining, Millipore Sigma, 46960) with a depth of around 50% of corneal stromal thickness (examined by optical coherence tomography (OCT), MICRON IV, Phoenix Technology Group). The occasional outliers were excluded from the experiments and were euthanized.

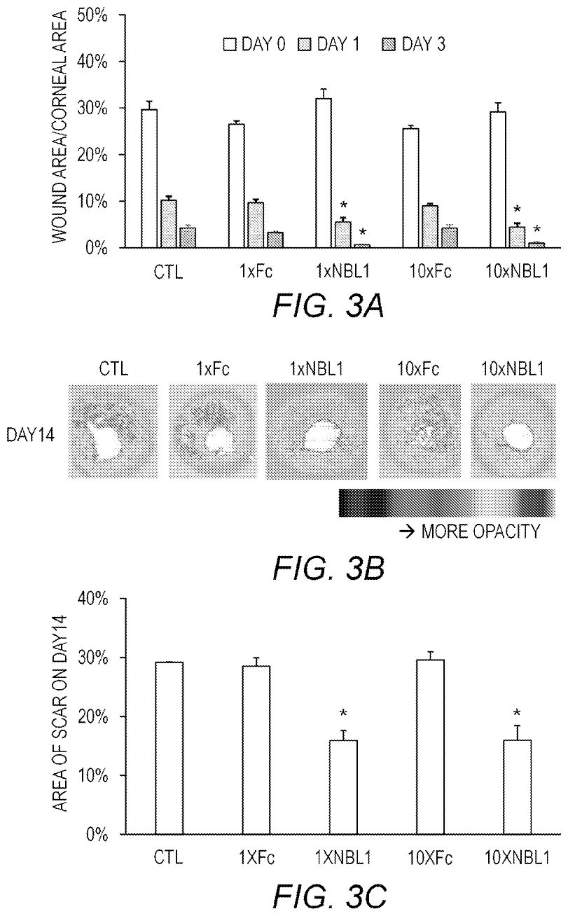

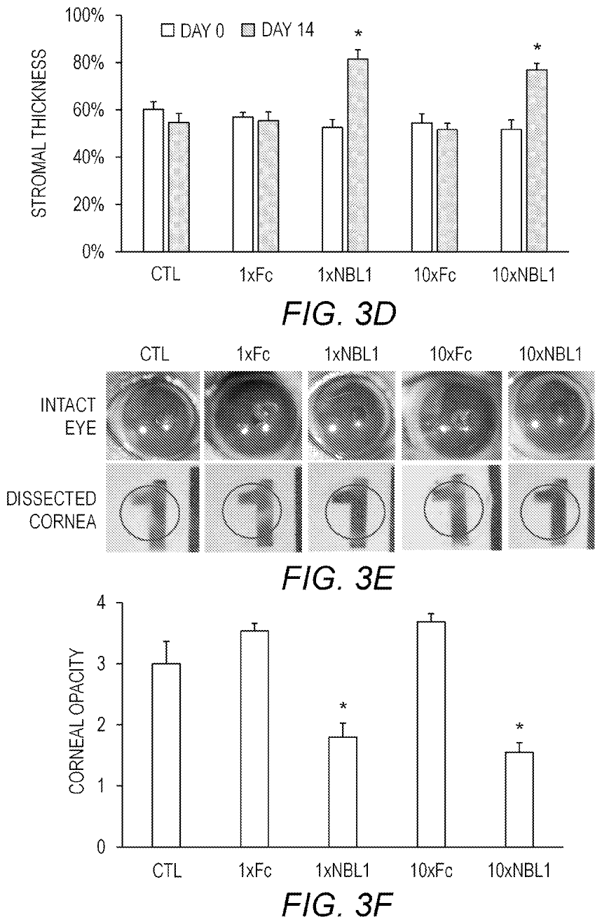

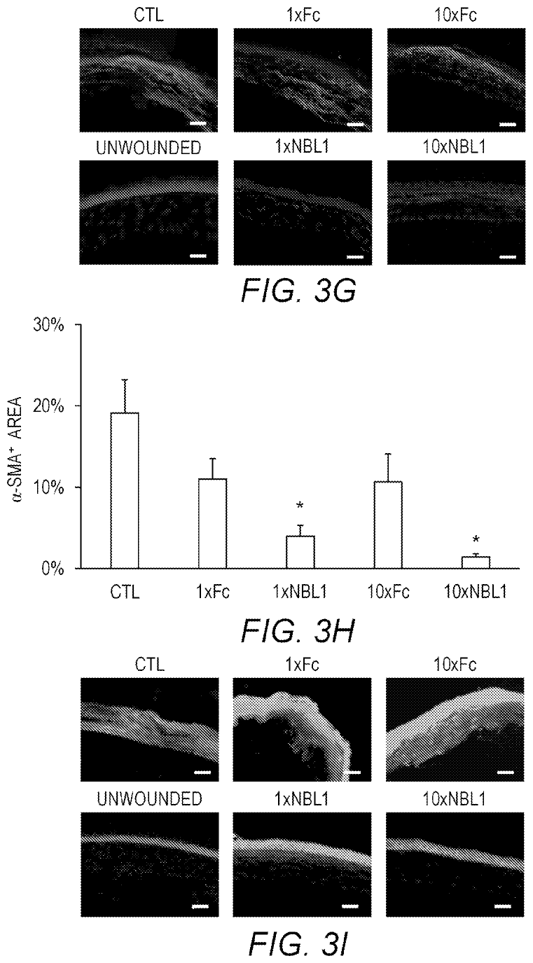

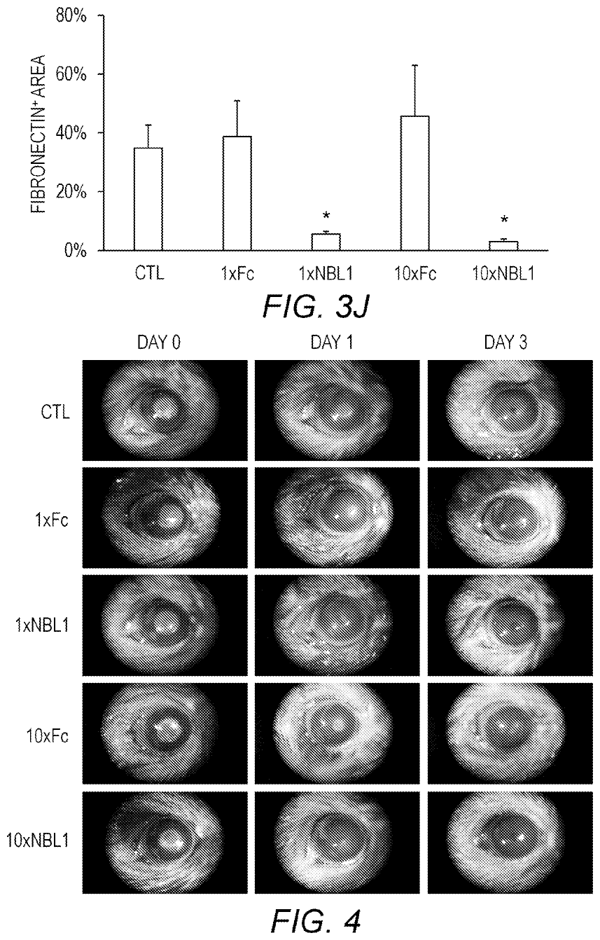

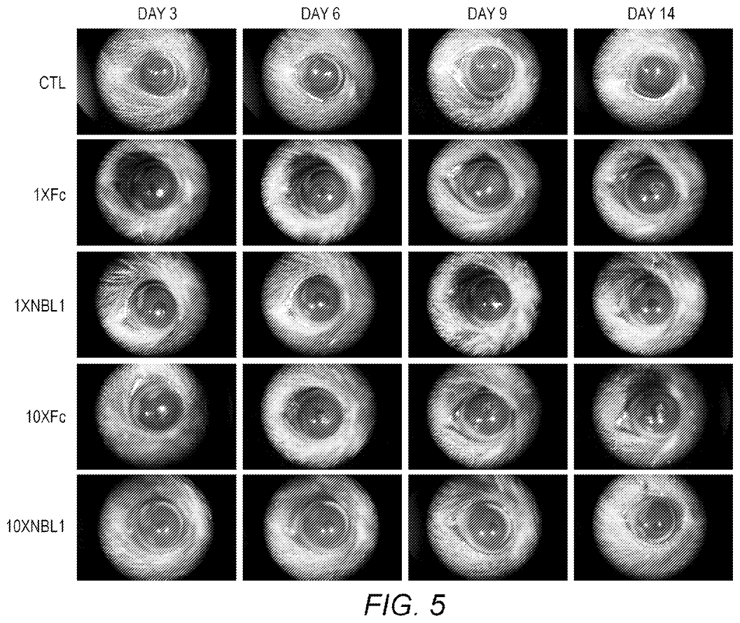

Data Collection and Results. During wound healing, wound re-epithelization was monitored by fluorescein staining which showed the size of the un-epithelialized wound. The NBL1-treated groups at both 1× and 10× doses showed a faster wound re-epithelization than Fe groups and vehicle control on Day 1 and Day 3 (quantitative analysis in FIG. 3A and representative pictures in FIG. 4). At the end of the 14-day treatment, the corneal scar was evaluated using 3 different methods: Optical coherence tomography (OCT, FIGS. 3B-3D), stereomicroscope (FIGS. 3E-3F), and protein expression (FIGS. 3G-3J). For Method 1 using in vivo evaluation by OCT, two sets of data were obtained: corneal reflectivity and corneal thickness. Yellow and red colors in OCT images indicate hyperreflectivity caused by opaque scars (FIG. 3B). Quantitative analysis of the hyperreflective areas in OCT images showed that NBL1 treated corneas at both 1× and 10× doses showed smaller hyperreflective scar areas than vehicle control and corresponding Fc controls (FIG. 3C). NBL1 treatment at both 1× and 10× doses showed a partial recovery of stromal thickness from around 52% on Day 0 to 78% on Day 14, while Ctl, 1×Fc, and 10×Fc did not show the recovery of stromal thickness (FIG. 3D), which is crucial to restore the mechanical strength of wounded corneas. There was no difference observed between 1×NBL1 and 10×NBL1. For Method 2 to evaluate the scar using stereomicroscope, intact eyes were observed (FIG. 3E, the process of scar formation during the 2-week study in FIG. 5) and dissected corneas placed above a printed number (number “1” on a ruler) (FIG. 3E, red circle indicates the edge of dissected cornea). Corneal opacity was graded by 3 blinded researchers with 4 grades: 1/clear, 2/slightly cloudy, 3/very cloudy, and 4/opaque. Corneas treated with NBL1 at both 1× and 10× doses were less opaque than vehicle control and corresponding Fc controls (FIG. 3F). For Method 3 to evaluate the scar by protein expression, severity of the scar was evaluated based on the presence of abnormal scar myofibroblast cells (α-SMA) and fibronectin expression in corneal stroma (FIG. 3G). NBL1 treatment significantly reduced corneal scar revealed by reduced α-SMA and fibronectin expression in corneal stroma (FIGS. 3G-3J). Unwounded mouse corneas were included as an additional control in every staining, which showed minimal expression of the three proteins in stroma (FIG. 3G). Results from the 3 different evaluation methods showed a consistent effect of NBL1 on reducing corneal scar formation after mechanical wounding in mice.

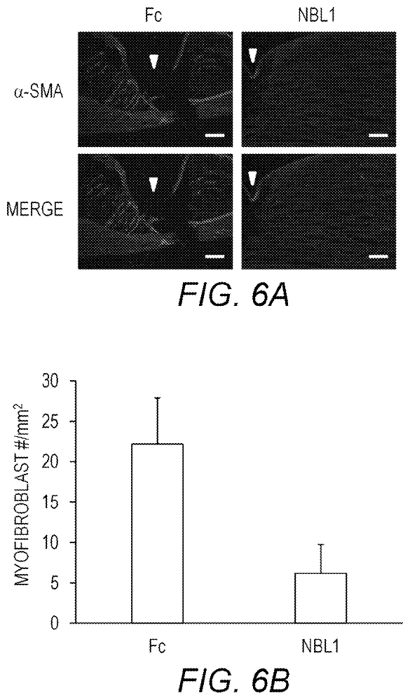

Function of NBL1 on human corneas during wound healing (organ culture model). After the anti-scar effect of NBL1 was confirmed in the mouse model, its effect was further examined on human samples using an organ culture model. The experiments were performed following published protocols (Sriram et al., Exp. Eye Res. 125:173 (2014); Castro et al., J. Vis. Exp. 2019(144)) with minor modifications. In brief, pairs of human corneas were obtained from the Eye Bank (Miracles In Sight) with the following criteria: (1) no corneal defect, and (2) death to delivery was within 4 days. The corneas were gently cleaned by removing blood residues, iris, and extra tenon and wounded by puncturing the central cornea using a 4 mm trephine to 90%-100% depth (the central corneal button was almost cut through but remained in position during culture). The wounded corneas were cultured submerged in DMEM medium supplemented with penicillin-streptomycin, gentamicin/amphotericin B, and 1 ng/ml TGF-β1 for 1 week. Both trephine cut and TGF-β1 treatment facilitated tissue fibrosis and scar formation. The experiment was conducted on 4 pairs of human corneas, of which the OS eyes were treated with NBL1 and the OD eyes were treated with Fc. The final concentration of NBL1 used in the medium was 0.3 μg/ml, which was “0.1×NBL1”. A reduced concentration of NBL1 was used here because of the following reason. Subconjunctival injection used in the mouse study produced a transient peak of a high drug concentration in cornea followed by a persistent long-time low drug concentration remaining in cornea (Baum et al., Ophthalmology 90(2):162 (1983)), which is estimated to be closer to 0.1×NBL1 when corneas were cultured constantly in medium. Culture medium was refreshed every 2 days. At the end of 1-week treatment, the cultured corneas were collected for cryosections. Wounded corneas treated with Fc (the same molar concentration as 0.1×NBL1, as a control group) showed obvious α-SMA expression, especially around the wound area, while the corneas treated with NBL1 showed a significantly reduced α-SMA expression (FIG. 6A). Quantitative analysis revealed that the NBL1-treated human corneas formed significantly fewer myofibroblasts (α-SMA⁺ cells, 6% in NBL1 group vs. 22% in Fc group) which are a major contributor to tissue fibrosis and scar formation (FIG. 6B). Data showed that NBL1 treatment reduced tissue fibrosis and scar formation in cultured human corneas under the scar-induced condition via mechanical wounding and TGF-β1.

### Example 2: Anti-Fibrotic Role of NBL1 in Other Organs

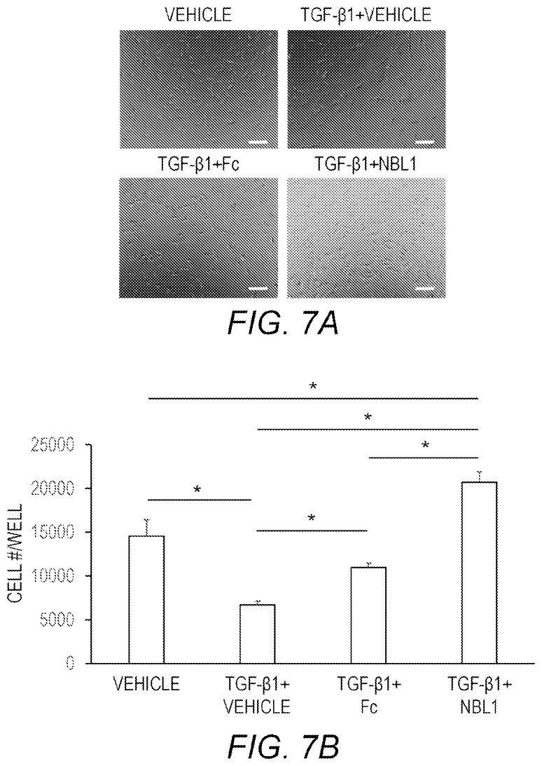

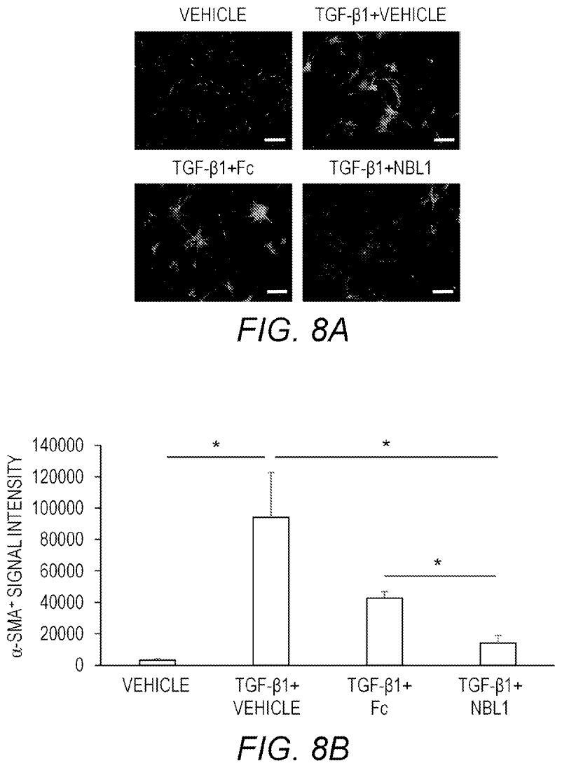

Primary lung fibroblasts from healthy human donors (obtained from the Marsico Lung Institute Tissue Procurement and Cell Culture Core at UNC-CH) were seeded in collagen-coated 24-wells plates and chamber slides at 10⁴ cells/well or chamber and incubated in serum-containing culture medium overnight for cell attachment. Then the cells were starved for 8 h in serum-free culture medium followed by different treatment groups: vehicle, TGF-β1 (1 ng/ml)+vehicle, TGF-β1 (1 ng/ml)+Fc (same molar concentration as NBL1), and TGF-β1+NBL1 (3 μg/ml) for 6 days. The medium was replaced every 2 days. At the end of the treatment, cells were collected, counted (FIGS. 7A-7B), and fixed for immunocytochemistry analysis (FIGS. 8A-8B). Under the stress of serum starvation, TGF-β1 caused fibroblast cell loss. However, NBL1 treatment rescued the cell loss caused by TGF-β1 under serum starvation (FIGS. 7A-7B), suggesting a role of NBL1 in preventing fibroblast loss during fibrosis. In addition, NBL1 treatment significantly inhibited the expression of α-SMA (myofibroblast marker, key player in tissue fibrosis and scar formation) induced by TGF-β1, indicating an anti-fibrotic effect of NBL1 in primary human lung fibroblast cells.

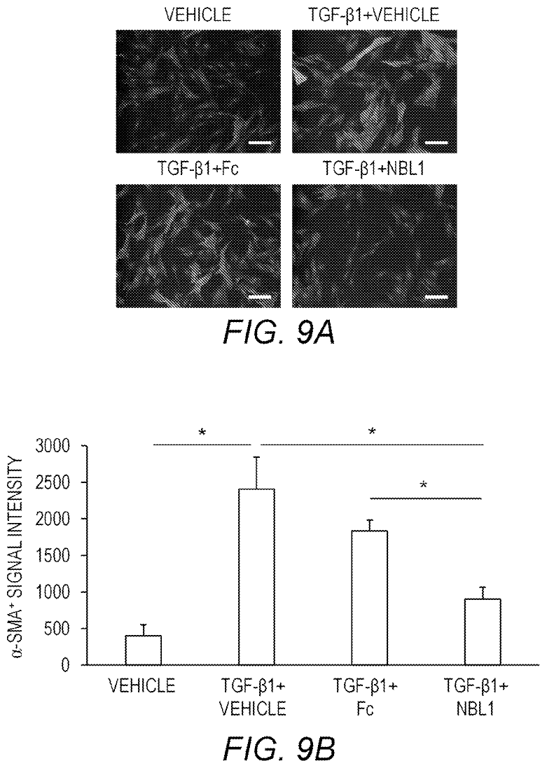

Primary dermal fibroblasts from healthy human donors (PCS-201-012, ATCC) were seeded in collagen-coated 24-wells plates and chamber slides at 3×10⁴ cells/well or chamber and incubated in serum-containing culture medium overnight for cell attachment. Then the cells were starved for 12 h in serum-free culture medium followed by different treatment groups: vehicle, TGF-β1 (10 ng/ml)+vehicle, TGF-β1 (10 ng/ml)+Fc (same molar concentration as NBL1), and TGF-β1+NBL1 (3 μg/ml) for 48 h. At the end of the treatment, cells were collected for immunocytochemistry analysis (FIGS. 9A-9B). NBL1 treatment significantly inhibited the expression of α-SMA (myofibroblast marker, key player in tissue fibrosis and scar formation) induced by TGF-β1, indicating an anti-fibrotic effect of NBL1 in primary human dermal fibroblast cells.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

1. A method for preventing or attenuating fibrosis and/or adhesion formation and/or scar formation in a subject in need thereof, the method comprising administering to the subject an effective amount of neuroblastoma suppressor of tumorigenicity 1 (NBL1), thereby preventing or attenuating fibrosis and/or adhesion formation and/or scar formation.

2. The method of claim 1, wherein the subject has a wound.

3. The method of claim 1, wherein the NBL1 is administered during or immediately after surgery, such as intra-abdominal surgery or intra-thoracic surgery.

4. (canceled)

5. The method of claim 1, wherein the NBL1 is administered during or immediately after insertion of a device or implant into the subject.

6. A method of inhibiting growth of an existing fibrosis, adhesion, or scar in a subject, comprising contacting the fibrosis, adhesion, or scar with an effective amount of NBL1, thereby inhibiting growth of the fibrosis, adhesion, or scar.

7. The method of claim 6, wherein the fibrosis, adhesion, or scar is contacted with NBL1 less than 1 month after fibrosis, adhesion, or scar formation, e.g., less than 3, 2, or 1 week.

8. The method of claim 6, wherein the fibrosis, adhesion, or scar is contacted with NBL1 once every 7 days, e.g., once every 6, 5, 4, 3, 2, or 1 day.

9. A method for treating, preventing, or inhibiting progression of a fibrotic disease in a subject in need thereof, the method comprising administering to the subject a composition comprising an effective amount of NBL1 to the site of fibrosis, thereby treating, preventing, or inhibiting progression of the fibrotic disease.

10. The method of claim 9, wherein the fibrotic disease is selected from the group consisting of fibrotic lung disease, hepatic fibrosis, rheumatoid arthritis, congestive heart failure, chronic renal disease, Lyme disease, stromal fibrosis, uterine fibroids, ovarian fibrosis, corneal fibrosis, and scarring.

11. (canceled)

12. The method of claim 10, wherein the fibrotic lung disease is selected from the group consisting of pulmonary fibrosis, fibrotic interstitial lung disease, interstitial pneumonia, fibrotic variant of non-specific interstitial pneumonia, cystic fibrosis, lung fibrosis, chronic obstructive pulmonary lung disease, and pulmonary arterial hypertension.

13. The method of claim 1, wherein the subject is a human.

14. The method of claim 1, wherein administering NBL1 or contacting the fibrosis, adhesion, or scar with NBL1 comprises delivering NBL1 protein to the subject.

15. The method of claim 1, wherein administering NBL1 or contacting the fibrosis, adhesion, or scar with NBL1 comprises delivering a nucleic acid encoding NBL1 to the subject.

16. The method of claim 1, wherein administering NBL1 or contacting the fibrosis, adhesion, or scar with NBL1 comprises delivering a cell recombinantly altered to express NBL1 to the subject.

17. The method of claim 1, wherein the NBL1 is administered systemically.

18. The method of claim 1, wherein the NBL1 is administered locally, e.g., to a wound or a site of fibrosis.

19. The method of claim 1, wherein the NBL1 is administered in an injectable composition, a topical composition, or a sprayable composition.

20-21. (canceled)

22. The method of claim 1, wherein the NBL1 is part of a fusion protein, such as a NBL1-Fc fusion protein.

23. (canceled)

24. A method of treating a corneal wound in a subject to facilitate wound re-epithelization and/or minimize scarring, or inhibiting scar formation in a corneal wound in a subject, comprising contacting the corneal wound with a therapeutically effective amount of NBL1, thereby facilitating wound re-epithelization.

25-46. (canceled)

47. A pharmaceutical composition comprising NBL1 protein, a nucleic acid encoding NBL1 protein, or a cell recombinantly altered to express NBL1 and a pharmaceutically acceptable carrier, wherein the pharmaceutical composition is suitable for ophthalmic administration.

48-54. (canceled)
